(19) United States
(12) Patent Application Publication
(10) Pub. No.: US 2026/0076788 A1
(43) Pub. Date: Mar. 19, 2026

(54) ORTHODONTIC COMPOSITION

(71) Applicant: Natalie DeVoir, Richland, WA (US)

(72) Inventor: Natalie DeVoir, Richland, WA (US)

(21) Appl. No.: 18/887,780

(22) Filed: Sep. 17, 2024

(51) Int. Cl. A61Q 11/00; A61C 15/04

(52) U.S. Cl. CPC A61C 15/041; A61Q 11/00

(57) ABSTRACT

An orthodontic composition is provided which may be for use in the mouth, such as a coating for dental floss and dental picks, and for use as an orthodontic wax composition or tooth wax composition. The orthodontic composition may include approximately: between 75.0% and 80.0% by weight beeswax, more preferably organic mechanically bleached beeswax; between 15.0% and 20.0% by weight coconut oil, more preferably organic coconut oil; and between 0.1% and 1.5% by weight xylitol, more preferably organic xylitol. Optionally, the composition 100 may comprise between 0.1% and 0.3% by weight natural flavors that preferably are organic. Preferably, the composition may be formed into an application body, the application body having a mass of between 1.0 and 10.0 grams. Preferably, an application body may include a convex surface and a concave surface, and the convex surface may be opposingly positioned relative to the concave surface.

### FIELD OF THE INVENTION

This patent specification relates to the field of orthodontic compositions. More specifically, this patent specification relates to an orthodontic wax composition, such as which may be applied to dental floss, dental picks and other instruments, and used as a tooth wax composition.

### BACKGROUND

Existing orthodontic wax on the marketplace is derived from crude oil (paraffin). A product of that origin should not be placed in the mouths of young, pre-pubescent and pubescent children. It should not be in adult bodies either. It is not technically marketed as ingestible, however it is inevitably being ingested by these children and by adults in braces.

Therefore, a need exists for novel orthodontic compositions that offer improved safety and tolerance from ingestion compared to existing orthodontic wax formulations. A further need exists for novel orthodontic compositions that are suitable for use as an orthodontic wax composition or tooth wax composition and which do not include ingredients that are derived from crude oil (paraffin).

### BRIEF SUMMARY OF THE INVENTION

An orthodontic composition is provided which may be for use in the mouth, such as an orthodontic wax composition or tooth wax composition. The composition may be used for a plurality of oral and dental applications, including: used as a coating for dental floss, applied to dental picks and other instruments, and used as an orthodontic wax or dental wax which can provide a temporary cover of sharp surfaces inside a user's mouth and help protect the mouth's soft tissues from the sharp surfaces.

In preferred embodiments, an orthodontic composition for use in the mouth may include approximately: between 75.0% and 80.0% by weight beeswax, more preferably organic mechanically bleached beeswax; between 15.0% and 20.0% by weight coconut oil, more preferably organic coconut oil; and between 0.1% and 1.5% by weight xylitol, more preferably organic xylitol. Optionally, the composition 100 may comprise between 0.01% and 0.5% by weight natural flavor, that preferably are organic.

In further embodiments, the composition may be formed into an application body, the application body having a mass of between 1.0 and 10.0 grams. Preferably, an application body may include a convex surface and a concave surface, and the convex surface may be opposingly positioned relative to the concave surface.

Numerous objects, features and advantages of the present invention will be readily apparent to those of ordinary skill in the art. Some example objects of the present invention are listed below.

One object of the present invention is to provide an orthodontic composition that has a limited number of ingredients that are all safe to ingest on their own or in combination. The ingredients preferably include organically sourced coconut oil, organically cultivated and mechanically bleached beeswax, and organically cultivated xylitol.

Another object is to provide an orthodontic composition that includes coconut oil which follows organic standards and allows orthodontic composition to be more malleable compared to existing orthodontic waxes, in order to place on sharp orthodontics brackets and to stay in place.

Another object is to provide an orthodontic composition which may be shaped into a novel application body that preferably may include a convex surface and a concave surface, and the convex surface may be opposingly positioned relative to the concave surface.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a top perspective view of an orthodontic composition formed into an example application body according to various embodiments described herein.

FIG. 2—FIG. 2 illustrates a bottom perspective view of an orthodontic composition formed into an example application body according to various embodiments described herein.

FIG. 3—FIG. 3 shows a sectional, through line 3-3 shown in FIG. 2, elevation view of the example application body of FIG. 2 according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

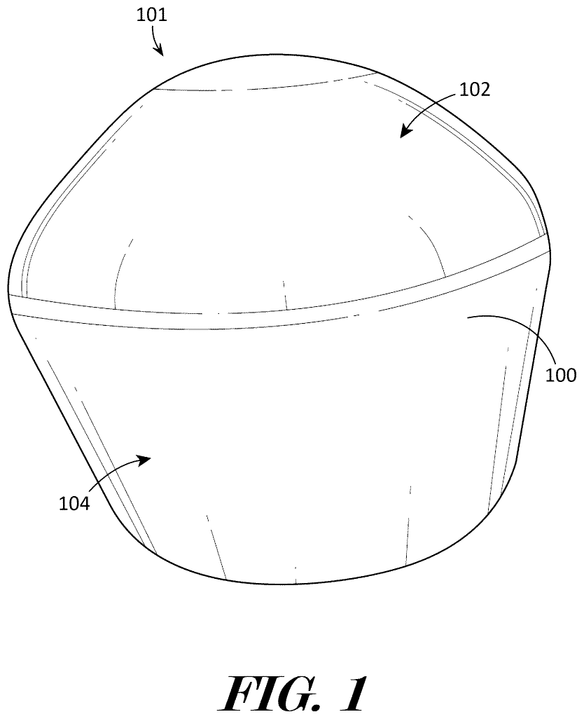

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new orthodontic composition that is suitable for use for a plurality of oral and dental applications, including: used as a coating for dental floss, applied to dental picks and other instruments, and for use as an orthodontic wax composition or tooth wax composition is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments. In some embodiments, an orthodontic composition (“the composition”) 100 is provided which may be for use in the mouth for a plurality of oral and dental applications, including: used as a coating for dental floss, applied to dental picks and other instruments, and for use as an orthodontic wax composition or tooth wax composition. Generally, orthodontic wax or dental wax is an excellent solution if one needs a comfortable, temporary fix to cover a protruding piece of a mouth appliance, such as braces or dentures. Orthodontic wax, also known as tooth wax, can provide a temporary cover of sharp surfaces inside a user's mouth and help protect the mouth's soft tissues from the sharp surfaces.

In preferred embodiments, an orthodontic composition 100 for use in the mouth may comprise approximately: between 75.0% and 80.0% by weight beeswax; between 15.0% and 20.0% by weight coconut oil; and between 0.1% and 1.5% by weight xylitol. Optionally, the composition 100 may comprise between 0.01% and 0.5% by weight natural flavor.

In some embodiments, the orthodontic composition 100 may comprise approximately between 75.0% and 80.0% by weight beeswax (preferably mechanically bleached beeswax), and more preferably, the orthodontic composition 100 may comprise approximately 75.0% and 80.0% by weight organic beeswax (preferably mechanically bleached organic beeswax). Beeswax (also known as cera alba) is a natural wax produced by honey bees of the genus Apis. In preferred embodiments, the beeswax of the composition 100 may comprise organically cultivated and mechanically bleached beeswax. Organic beeswax is beeswax that is processed without harmful chemicals or agents that could alter its properties. It is usually certified by the USDA to ensure that the raw ingredients are processed according to strict standards. Mechanically bleached beeswax is a type of white beeswax that is refined without the use of chemicals. It is stable, non-yellowing, and free of impurities. Beeswax that has been bleached mechanically has been filtered and re-filtered until it is no longer that warm yellow color (the more white it is the more aesthetic and similar to the teeth color when placed over the orthodontic bracket).

In further embodiments, the orthodontic composition 100 may comprise between 77.0% and 80.0% by weight beeswax (preferably mechanically bleached beeswax), and more preferably, the orthodontic composition 100 may comprise between 77.0% and 80.0% by weight organic beeswax (preferably mechanically bleached organic beeswax). In further embodiments, the orthodontic composition 100 may comprise between 78.7% and 80.0% by weight beeswax (preferably mechanically bleached beeswax), and more preferably, the orthodontic composition 100 may comprise between 78.7% and 80.0% by weight organic beeswax (preferably mechanically bleached organic beeswax). In further embodiments, the orthodontic composition 100 may comprise between 79.0% and 79.4% by weight beeswax (preferably mechanically bleached beeswax), and more preferably, the orthodontic composition 100 may comprise approximately between 79.0% and 79.4% by weight organic beeswax (preferably mechanically bleached organic beeswax). In further embodiments, the orthodontic composition 100 may comprise substantially 79.2% by weight beeswax (preferably mechanically bleached beeswax), and more preferably, the orthodontic composition 100 may comprise substantially 79.2% by weight organic beeswax (preferably mechanically bleached organic beeswax).

In some embodiments, the orthodontic composition 100 may comprise approximately 15.0% and 20.0% by weight coconut oil, and more preferably, the orthodontic composition 100 may comprise approximately 15.0% and 20.0% by weight organic coconut oil. Coconut oil (or coconut fat) is an edible oil derived from the kernels, meat, and milk of the coconut palm fruit. Coconut oil is a white solid fat below around 25° C. (77° F.), and a clear thin liquid oil in warmer climates. Unrefined varieties have a distinct coconut aroma. Coconut oil is used as a food oil, and in industrial applications for cosmetics and detergent production. The oil is rich in medium-chain fatty acids. In some embodiments, the coconut oil of the composition 100 may comprise refined, bleached, and deodorized (RBD) coconut oil that is usually made from copra and dried coconut kernels, which are pressed in a heated hydraulic press to extract the oil. Unlike virgin coconut oil, refined coconut oil has no coconut taste or aroma. RBD oil is used for home cooking, commercial food processing, and cosmetic, industrial, and pharmaceutical purposes.

In preferred embodiments, the coconut oil of the composition 100 may comprise organically sourced coconut oil that follows organic standards that preferably has USDA Organic Certification. Organic or pure coconut oil is extracted straight from the fresh coconut meat rather than dried. The coconut meat which is utilized for making organic coconut oil mainly undergoes two processes like quick drying or wet milling. Organic coconut oil is made from plants that are grown without harmful chemicals, such as pesticides, synthetic fertilizers, herbicides, and GMOs. This is the only government-regulated system that verifies the safety of organic coconut oil products.

In further embodiments, the orthodontic composition 100 may comprise between 18.0% and 20.0% by weight coconut oil, and more preferably, the orthodontic composition 100 may comprise between 18.0% and 20.0% by weight of organic coconut oil. In further embodiments, the orthodontic composition 100 may comprise between 19.3% and 20.0% by weight coconut oil, and more preferably, the orthodontic composition 100 may comprise between 19.3% and 20.0% by weight organic coconut oil. In further embodiments, the orthodontic composition 100 may comprise between 19.6% and 20.0% by weight coconut oil, and more preferably, the orthodontic composition 100 may comprise between 19.6% and 20.0% by weight organic coconut oil. In further embodiments, the orthodontic composition 100 may comprise substantially 19.8% by weight coconut oil, and more preferably, the orthodontic composition 100 may comprise substantially 19.8% by weight organic coconut oil.

In some embodiments, the orthodontic composition 100 may comprise approximately between 0.1% and 1.5% by weight xylitol, and more preferably, the orthodontic composition 100 may comprise approximately between 0.1% and 1.5% by weight organic xylitol. Xylitol is a white, crystalline substance and is a sugar alcohol, or polyol, that occurs naturally in fruits and vegetables, and is also produced from corn cobs and birch bark. Xylitol is a chemical compound with the formula C₅H₁₂O₅, and is classified as a polyalcohol and a sugar alcohol, specifically an alditol. Xylitol reduces plaque formation and bacterial adherence (i.e., is antimicrobial), inhibits enamel demineralization (i.e., reduces acid production), and has a direct inhibitory effect on mutans streptococci (MS) bacteria. In preferred embodiments, the xylitol of the composition 100 may comprise organic xylitol that is derived from organic, non-GMO corn. In further preferred embodiments, the xylitol of the composition 100 may comprise USDA Certified Organic Xylitol.

In further embodiments, the orthodontic composition 100 may comprise between 0.3% and 1.3% by weight xylitol, and more preferably, the orthodontic composition 100 may comprise between 0.3% and 1.3% by weight organic xylitol. In further embodiments, the orthodontic composition 100 may comprise between 0.5% and 1.1% by weight xylitol, and more preferably, the orthodontic composition 100 may comprise between 0.5% and 1.1% by weight organic xylitol. In further embodiments, the orthodontic composition 100 may comprise between 0.75% and 0.85% by weight xylitol, and more preferably, the orthodontic composition 100 may comprise between 0.75% and 0.85% by weight organic xylitol. In further embodiments, the orthodontic composition 100 may comprise substantially 0.8% by weight xylitol, and more preferably, the orthodontic composition 100 may comprise substantially 0.8% by weight organic xylitol.

In some embodiments, the orthodontic composition 100 may comprise approximately between 0.01% and 0.5% by weight natural flavor. Natural flavor, also called naturally derived flavoring, may comprise any flavoring agent suitable for use by humans that comes from a plant or animal source, and is used to enhance the taste of food and drinks. Natural flavors can be extracted from a variety of sources, including: fruits, vegetables, and fruit or vegetable juices, spices and herbs, roots, bark, buds, and leaves. Natural flavors can be manipulated in a lab through distillation, fermentation, or other methods. For example, vanilla flavor is extracted from vanilla beans using alcohol extraction, while fruit flavors are obtained through pressing or enzyme-assisted extraction. Natural flavors are different from artificial flavors, which are made using man-made chemicals. Example natural flavor that may be incorporated into the composition 100 may include mint, clove-orange (used in dentistry to help heal a sore in the oral cavity), lemon, maple syrup, vanilla, sweet and sour, sour and spicy, etc.

In further embodiments, the orthodontic composition 100 may comprise between 0.1% and 0.3% by weight natural flavor. In further embodiments, the orthodontic composition 100 may comprise between 0.1% and 0.3% by weight natural flavor. In further embodiments, the orthodontic composition 100 may comprise substantially 0.2% by weight natural flavor.

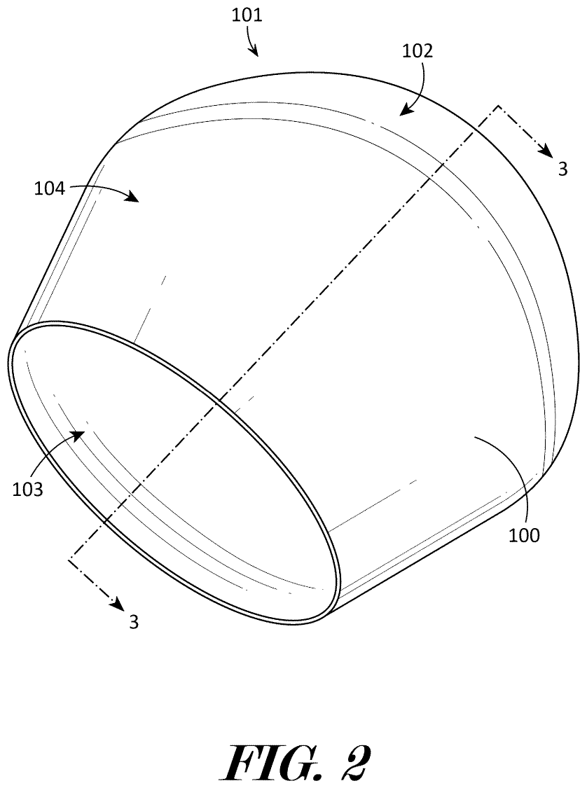

The composition 100 may be formed in any shape and size. In preferred embodiments, and referring to FIGS. 1-3, the composition 100 may be formed into an application body 101, in which the application body 101 may have a mass of between 1.0 to 10.0 grams. An application body 101 may comprise a preformed or pre-shaped unit of the composition 100 that a user may apply to a desired surface or object in their mouth. Optionally, an application body 101 may be dusted with xylitol powder, such as within a packaging tin or container having a plurality of application bodies 101 to help avoid the application bodies 101 from melting/sticking together.

In some embodiments, an application body 101 may have a convex surface 102. Generally, a convex surface 102 may comprise a surface that is curved or rounded outward from the application body 101, like the outside of a sphere or circle. In some embodiments, a convex surface 102 may be configured in the shape of a spherical cap that is the region of a sphere which lies above (or below) a given plane. In some embodiments, a convex surface 102 may be configured in the shape of a hemisphere or half of a sphere.

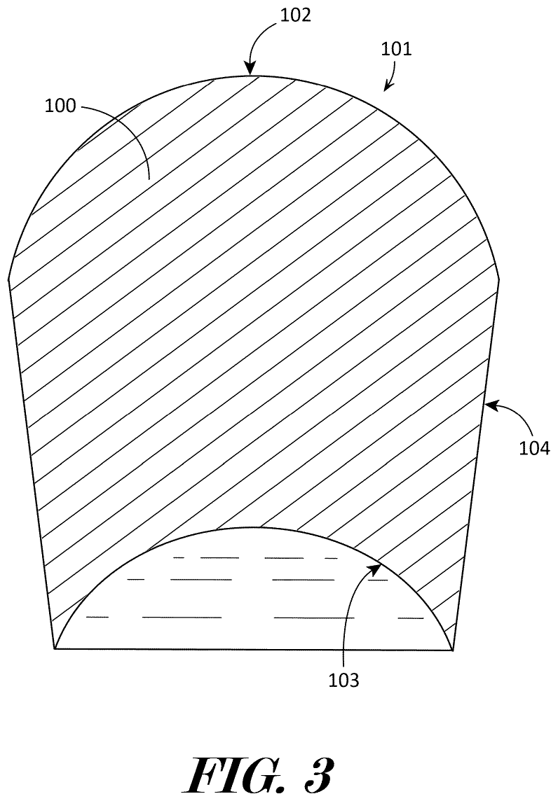

In some embodiments, an application body 101 may have a concave surface 103. Generally, a convex surface 102 may comprise a surface that curves inward into the application body 101, or is hollowed out or rounded inward. In some embodiments, a concave surface 103 may be configured in the shape of a spherical cap that is the region of a sphere which lies above (or below) a given plane. In some embodiments, a concave surface 103 may be configured in the shape of a hemisphere or half of a sphere. In preferred embodiments, the application body 101 may comprise a convex surface 102 that is opposingly positioned relative to the concave surface 103 as perhaps best shown in FIG. 3.

In some embodiments, an application body 101 may have a bridge surface 104. In preferred embodiments, a bridge surface 104 may extend between a convex surface 102 and a concave surface 103 so that the convex surface 102 and the concave surface 103 may both be generally coupled to the bridge surface 104. In some embodiments, a bridge surface 104 may be positioned between a convex surface 102 and a concave surface 103 so that the convex surface 102 and the concave surface 103 may both be separated by the bridge surface 104. In some embodiments, a bridge surface 104 may comprise a cylindrical shape. In some embodiments, bridge surface 104 may comprise a cone frustum or frustum shape. A frustum, also called a truncated cone, is created when a cone is cut by a plane parallel to its base, leaving the bottom part of the cone as the frustum. A frustum has two circular bases, one smaller than the other, connected by a curved surface (the bridge surface 104 functioning as the curved surface of the frustum).

While some exemplary shapes and sizes have been provided for an application body 101, it should be understood to one of ordinary skill in the art that an application body 101 may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An orthodontic composition for use in the mouth, the composition comprising:
a. between 75.0% and 80.0% by weight beeswax;
b. between 15.0% and 20.0% by weight coconut oil; and
c. between 0.1% and 1.5% by weight xylitol.

2. The composition of claim 1, wherein the beeswax comprises mechanically bleached beeswax.

3. The composition of claim 1, wherein the beeswax comprises between 77.0% and 80.0% by weight.

4. The composition of claim 1, wherein the beeswax comprises between 78.7% and 80.0% by weight.

5. The composition of claim 1, wherein the coconut oil comprises between 18.0% and 20.0% by weight.

6. The composition of claim 1, wherein the coconut oil comprises between 19.3% and 20.0% by weight.

7. The composition of claim 1, wherein the xylitol comprises between 0.3% and 1.3% by weight.

8. The composition of claim 1, wherein the xylitol comprises between 0.5% and 1.1% by weight.

9. The composition of claim 1, further comprising between 0.01% and 0.5% by weight natural flavor.

10. The composition of claim 1, further comprising between 0.1% and 0.3% by weight natural flavor.

11. The composition of claim 1, wherein the composition is formed into an application body, the application body having a mass of between 1.0 and 10.0 grams.

12. The composition of claim 11, wherein the application body comprises a convex surface.

13. The composition of claim 12, wherein the application body comprises a concave surface.

14. The composition of claim 13, wherein the convex surface is opposingly positioned relative to the concave surface.

15. An orthodontic composition for use in the mouth, the composition comprising:
a. between 77.0% and 80.0% by weight organic beeswax that is mechanically bleached;
b. between 18.0% and 20.0% by weight organic coconut oil; and
c. between 0.3% and 1.3% by weight xylitol.

16. The composition of claim 15, further comprising between 0.01% and 0.5% by weight natural flavor.

17. The composition of claim 15, further comprising between 0.1% and 0.3% by weight natural flavor.

18. The composition of claim 15, wherein the composition is formed into an application body, the application body having a mass of between 1.0 and 10.0 grams.

19. The composition of claim 18, wherein the application body comprises a convex surface.

20. The composition of claim 19, wherein the application body comprises a concave surface, and wherein the convex surface is opposingly positioned relative to the concave surface.
